(19) United States
(12) Patent Application Publication
(10) Pub. No.: US 2007/0110786 A1
(43) Pub. Date: May 17, 2007

(54) Medical articles having enhanced therapeutic agent binding

(75) Inventors: Barron Tenney, Haverhill, MA (US); Michael N. Helmus, Worcester, MA (US)

Correspondence Address: MAYER & WILLIAMS PC, 251 NORTH AVENUE WEST, 2ND FLOOR, WESTFIELD, NJ, 07090, US

(73) Assignee: Boston Scientific Scimed, Inc.

(21) Appl. No.: 11/274,538

(22) Filed: Nov. 15, 2005

(51) Int. Cl. A61K 48/00; A61F 2/02

(52) U.S. Cl. 424/423; 514/44

(57) ABSTRACT

According to an aspect of the present invention, medical articles are provided which comprise the following (a) a polymeric region having a first charge, and (b) a charged therapeutic agent having a second charge that is opposite in sign to that of the first charge. In certain beneficial embodiments, the medical articles are high surface area articles, for example, articles formed using small diameter (e.g., 10 microns or less) fibers.

### FIELD OF THE INVENTION

This invention relates to medical articles, and more particularly to medical articles that contain one or more therapeutic agents.

### BACKGROUND OF THE INVENTION

The in vivo delivery of therapeutic agents to patients' bodies is common in the practice of modern medicine. In vivo delivery of therapeutic agents is often implemented using medical devices that may be temporarily or permanently placed at a target site within the body. These medical devices can be maintained, as required, at their target sites for short or prolonged periods of time, delivering therapeutic agents at the target sites.

In accordance with some delivery strategies, a therapeutic agent is provided within or beneath a polymeric layer that is associated with the medical device. Once the medical device is placed at the desired location within a patient, the therapeutic agent is released from the medical device with a profile that is dependent, for example, upon the loading of the therapeutic agent and upon the nature of the polymeric layer. In many such instances, however, the therapeutic agent remains trapped within the medical device and is thus of no benefit to the patient.

One solution to this problem is to dispose the therapeutic agent at the surface of the medical device. However, the amount of therapeutic agent that can be disposed at the surface is limited, particularly for smooth, planar surfaces. Moreover, without significant attractive forces between the therapeutic agent and the surface, minimal agent may ultimately become bound to the surface and/or the agent will be released prematurely in the body, resulting in a loss in efficacy for the therapeutic agent.

These and other drawbacks are addressed by the present invention.

### SUMMARY OF THE INVENTION

According to an aspect of the present invention, medical articles are provided which comprise the following (a) a polymeric region having a first charge, and (b) a charged therapeutic agent having a second charge that is opposite in sign to that of the first charge. In certain beneficial embodiments, the medical articles are high surface area articles, for example, articles formed using small diameter (e.g., 10 microns or less) fibers.

An advantage of the present invention is that the amount of therapeutic agent that is bound to the medical articles of the present invention may be enhanced.

Another advantage of the present invention is that the amount of therapeutic agent that is released prematurely may be decreased.

These and other embodiments and advantages of the present invention will become immediately apparent to those of ordinary skill in the art upon review of the Detailed Description and Claims to follow.

### DETAILED DESCRIPTION OF THE INVENTION

According to an aspect of the present invention, medical articles are provided which contain at least one polymeric region and at least one therapeutic agent. The polymeric regions and therapeutic agents selected for use in the articles of the present invention are of opposite charge, thereby promoting electrostatic binding between the therapeutic agent and the surface of the polymeric region.

A positive charge may arise, for example, from the presence of cationic moieties. Anionic moieties may also be present, so long as the anionic moieties contribute less to the overall charge than do the cationic moieties.

Conversely, a negative charge may arise, for example, from the presence of anionic moieties. Cationic moieties may also be present, so long as the cationic moieties contribute less to the overall charge than do the anionic moieties.

The charge of a particular polymeric region or therapeutic agent may change with the pH of its surrounding environment. Consequently, the pH encountered by the polymeric region and the therapeutic agent at the time of binding may be monitored and controlled in some embodiments to optimize the charge differential between the polymeric material and the therapeutic agent. In certain embodiments, efforts are made to match the binding pH with the physiological pH experienced by the medical article within the body, for example, in order to enhance binding continuity from environment to environment. On the other hand, in certain embodiments, the physiological pH may differ substantially from the binding pH, such that the therapeutic agent and/or the polymeric region may become more charge neutral, or such that the charge of the therapeutic agent and/or the polymeric region changes sign (e.g., in the case of therapeutic agents or polymeric regions having both acidic and basic functional groups), resulting in rapid release of the therapeutic agent.

The present invention is applicable to a wide range of medical articles including, for example, internal medical devices (e.g., medical devices that are at least partially implantable, insertable, etc.). Internal medical devices benefiting from the various aspects and embodiments of the present invention are numerous and may be selected, for example, from the following: catheters (e.g., renal or vascular catheters such as balloon catheters), balloons, guide wires, filters (e.g., vena cava filters), stents (including coronary vascular stents, peripheral vascular stents, cerebral, urethral, ureteral, biliary, tracheal, gastrointestinal and esophageal stents), stent grafts, vascular grafts, vascular access ports, embolization devices including cerebral aneurysm filler coils (such as Guglilmi detachable coils and various other metal coils), myocardial plugs, septal defect closure devices, patches, pacemakers and pacemaker leads, defibrillation leads and coils, left ventricular assist hearts and pumps, total artificial hearts, heart valves, vascular valves, tissue engineering scaffolds for in vivo tissue regeneration, biopsy devices, as well as many other devices that are implanted or inserted into the body and from which therapeutic agent is released.

Polymeric regions for use in the various aspects and embodiments of the present invention may be provided in a variety of forms, including polymeric layers that are formed over all or only a portion of an underlying medical article substrate, polymeric regions that do not require an underlying substrate, such as scaffolds and fibers, and so forth.

Layers can be provided over an underlying substrate at a variety of locations, and in a variety of shapes. They may be stacked on one another. Consequently, one can stack multiple layers each having its own bound therapeutic agent, such that the therapeutic agents emerge in series. As used herein a “layer” of a given material is a region of that material whose thickness is small compared to both its length and width. As used herein a layer need not be planar, for example, taking on the contours of an underlying substrate. Layers can be discontinuous (e.g., patterned). Terms such as “film,” “layer” and “coating” may be used interchangeably herein. Where polymeric layers are formed over all or only a portion of an underlying substrate, the underlying substrate may be formed from a variety of materials including metallic materials and non-metallic materials such as ceramic materials, carbon-based materials, silicon-based materials, polymeric materials, and so forth.

Where fibers are employed, the polymer region may correspond, for example, to the entire fiber or to a coating on the fiber. Medical articles in accordance with the present invention that are formed from fibers include two-dimensional structures (e.g., patches) and three-dimensional structures (e.g., tubes), which may be formed using any suitable fiber-based construction technique including, for example, a variety of woven and non-woven techniques. Examples of non-woven techniques include those utilizing thermal fusion, fusion due to removal of residual solvent, mechanical entanglement, chemical binding, adhesive binding, and so forth. Moreover, the polymer region may correspond to a fiber-containing region (e.g., a woven or non-woven fiber-containing region) that is disposed over a substrate, for example, a medical device substrate (e.g., a metallic stent substrate such as a stainless steel or nitinol stent substrate), among many other possibilities.

Specific examples of fiber-based medical articles include hollow fibers for oxygenators, patches (including replacement patches) such as patches for hernia repair and patches for the gastrointestinal tract and the urogenital system, fabric to join LVADs (left ventricular assist devices) and TAHs (total artificial hearts) to human arteries, wound dressings, membranes, anterior cruciate ligaments, neurovascular aneurysm treatment articles, valve leaflets for heart valves and venous valves, grafts, including large and small vascular grafts such as coronary artery bypass grafts, peripheral vascular grafts and endovascular grafts, stent grafts, grafts for the gastrointestinal tract and the urogenital system, vascular access devices including vascular access ports and arterio-venous access grafts (e.g., devices which are utilized to give frequent arterial and/or venous access such as for antibiotics, total parental nutrition, intravenous fluids, blood transfusion, blood sampling, or arterio-venous access for hemodialysis, and so forth), other tubular structures, for example, biliary, urethral, ureteral and uterine tubular structures, embolic filters, scaffolds for tissue engineering including cardiac tissue, skin, mucosal and vascular tissue, and so forth.

Regardless of their overall form, polymeric regions suitable for use in the present invention are beneficially high surface area regions. For instance, where the polymeric regions are provided in the form of fibers or fiber coatings, as a general rule (e.g., assuming no variation in surface texture), decreases in fiber diameter will lead to increases in surface area of the polymer regions. Thus, fibers selected for use in the present invention include small diameter fibers, which have diameters that are less than 10 microns (μm), less than 1 micron, or even less than 500 nm (e.g., 10 nm to 25 nm to 50 nm to 100 nm to 250 nm to 500 nm). Taking as an example a polymer having a density of 1 g/cm³, 1 gram of that polymer corresponds to 1 cubic centimeter of material. For a smooth fiber, fiber volume is equal to πr²L. For a 1 micron fiber, which has a radius of 0.5×10⁴ cm, 1 cm³ of polymer=π(0.5×10⁴ cm)²L, which corresponds to a fiber length of 1.2732×10⁸ cm. The surface area of this fiber (excluding end area, which is insignificant) is πdL=π(10⁴ cm)(1.273×10⁸ cm)=4×10⁴ cm², which corresponds to a specific surface area of 4×10⁴ cm²/g. Please note that surface area is inversely proportional to diameter. For example, a 10 micron fiber by this calculation has a specific surface area of 4×10³ cm²/g, whereas a 0.1 micron fiber has a specific surface area of 4×10⁵ cm²/g. Hence, specific surface areas for fibers in accordance with the present invention may range, for example, from 10³ cm²/g to 10⁴ cm²/g to 10⁵ cm²/g, among other values.

In addition to decreasing material cross section (e.g., diameter, in the case of fibers), surface area may also be increased through the creation of surface texture. In some embodiments, the polymeric regions employed for the practice of the present invention have nanotextured surfaces. Nanotextured surfaces are those that contain surface nanofeatures, which are surface features (e.g., raised features, depressed features, etc.) that have at least one dimension (and often two or three dimensions) less than 100 nm in length. As a specific example, it is noted that a ridge or trench that is 10 nm wide, by 10 nm high/deep, by 1 micron long is nonetheless a nanostructure, as the term is used herein, because it has at least one dimension (i.e., its width and its height/depth), which is less than 100 nm in length. Of course, nanotextured surfaces may also contain features that are not nanofeatures. Beside ridges and trenches, other examples of surface nanofeatures include hills, mesas/plateaus, terraces, surface pores, and so forth.

A “polymeric region” is a region that contains polymers, commonly at least 50 wt %, 75 wt %, 90 wt %, 95 wt % or even more, polymers. As used herein, “polymers” are molecules that contain multiple copies of one or more constitutional units, commonly referred to as monomers, and typically containing from 5 to 10 to 25 to 50 to 100 to 500 to 1000 or more constitutional units. The polymers may be, for example, homopolymers, which contain multiple copies of a single constitutional unit, and/or copolymers, which contain multiple copies of at least two dissimilar constitutional units, which units may be present in any of a variety of distributions including random, statistical, gradient, and periodic (e.g., alternating) distributions. The polymers for use in the present invention may have a variety of architectures, including cyclic, linear and branched architectures. Branched architectures include star-shaped architectures (e.g., architectures in which three or more chains emanate from a single branch point), comb architectures (e.g., architectures having a main chain and a plurality of side chains) and dendritic architectures (e.g., arborescent and hyperbranched polymers), among others. “Block copolymers” are polymers containing two or more differing polymer chains, for example, selected from homopolymer chains and random and periodic copolymer chains.

As noted above, polymeric regions in accordance with the present invention may be negatively or positively charged, for example, (a) by forming the polymeric regions using polymers that are either inherently charged or are modified to possess a charge, or (b) by modifying the polymeric regions to introduce a surface charge after they are formed.

Examples of polymers that are inherently charged include polyionic polymers that are polycationic and those that are polyanionic at a relevant pH (e.g., the binding pH). Such polymers typically have multiple (e.g., 5, 10, 25, 50, 100, or more, frequently, many more) charged sites. They include polyacids, polybases and polysalts, for example, polyelectrolytes, including ionomers (polyelectrolytes in which a small but significant proportion of the constitutional units carry charges). As indicated above, polymers containing both cationic and anionic groups are categorized herein as either polycationic polymers or polyanionic polymers, depending on the relative amounts of anionic and cationic groups possessed by the polymer.

Polycationic polymers suitable for the practice of the invention may be natural or synthetic, they may be homopolymers or copolymers, and they may be used singly or in blends. Polycationic polymers for the practice of the present invention may be selected, for example, from suitable homopolymers and copolymers that have or are capable of having (e.g., via protenation or salt dissociation), one or more of the following cationic groups: charged amino groups, including charged primary (—NH₃⁺), secondary and tertiary amino groups, amidinium groups, guanidinium groups, triazolium groups, imidazolium groups, imidazolinium groups, pyridinium groups, sulfonium groups, including primary (—SH₂⁺) and secondary sulfonium groups, hydrosulfide groups, phosphonium groups, including primary (—PH₃⁺), secondary, and tertiary phosphonium groups, isothiouronium groups, nitrosyl groups, nitryl groups, tropilium groups, iodonium groups, arsonium groups, antimonium groups, oxonium groups, and anilinium groups, among others.

Specific examples of polycationic polymers may be selected, for example, from suitable members of the following: polyamines, including polyamidoamines, poly(amino methacrylates) including poly(dialkylaminoalkyl methacrylates) such as poly(dimethylaminoethyl methacrylate) and poly(diethylaminoethyl methacrylate), polyvinylamines, polyvinylpyridines including quaternary polyvinylpyridines such as poly(N-ethyl-4-vinylpyridine), poly(vinylbenzyltrimethylamines), polyallylamines and poly(diallyldialklylamines) such as poly(diallyldimethylammonium chloride), spermine, spermidine, hexadimethrene bromide (polybrene), polyimines including polyalkyleneimines such as polyethyleneimines, polypropyleneimines and ethoxylated polyethyleneimines, basic peptides and proteins, including histone polypeptides and polymers containing lysine, arginine, ornithine and combinations thereof including poly-L-lysine, poly-D-lysine, poly-L,D-lysine, poly-L-arginine, poly-D-arginine, poly-D,L-arginine, poly-L-ornithine, poly-D-ornithine, poly-L,D-ornithine, gelatin, albumin, protamine, and polycationic polysaccharides such as cationic starch and chitosan, among others.

As with polycationic polymers, polyanionic polymers suitable for the practice of the invention may be natural or synthetic, they may be homopolymers or copolymers, and they may be used singly or in blends. Polyanionic polymers for the practice of the present invention may be selected, for example, from suitable homopolymers and copolymers that have or are capable of having (e.g., via proton donation or salt dissociation), one or more of the following anionic groups: phosphate groups, sulfate groups, sulfonate groups, phosphonates groups and carboxylate groups, among others.

Specific examples of polyanionic polymers may be selected, for example, from suitable members of the following: (a) polysulfonates such as polyvinylsulfonates, poly(styrenesulfonates) such as poly(sodium styrenesulfonate) (PSS), sulfonated poly(tetrafluoroethylene), sulfonated polymers such as those described in U.S. Pat. No. 5,840,387, including sulfonated styrene-ethylene/butylene-styrene triblock copolymers, sulfonated styrenic homopolymers and copolymer such as a sulfonated versions of the polystyrene-polyolefin copolymers described in U.S. Pat. No. 6,545,097 to Pinchuk et al., which polymers may be sulfonated, for example, using the processes described in U.S. Pat. No. 5,840,387 and U.S. Pat. No. 5,468,574, as well as sulfonated versions of various other homopolymers and copolymers, (b) polycarboxylates such as acrylic acid polymers and salts thereof (e.g., ammonium, potassium, sodium, and so forth salts), for instance, those available from Atofina and Polysciences Inc., methacrylic acid polymers and salts thereof (e.g., EUDRAGIT, a methacrylic acid and ethylacrylate copolymer), carboxymethylcellulose, carboxymethylamylose and carboxylic acid derivatives of various other polymers, polyanionic peptides and proteins such as glutamic acid polymers and copolymers, aspartic acid polymers and copolymers, and gelatin, (c) polyphosphates such as phosphoric acid derivatives of various polymers, (d) polyphosphonates such as polyvinylphosphonates, (e) polysulfates such as polyvinylsulfates, and so forth.

Additional examples include sulfated and non-sulfated polysaccharides, including sulfated and non-sulfated glycosaminoglycans as well as species containing the same such as proteoglycans, for instance, selected from heparin, heparin sulfate, chondroitin sulfate, keratan sulfate, dermatan sulfate, hyaluronan, bamacan, perlecan, biglycan, fibromodulin, aggrecan, decorin, mucin, carrageenan, polymers and copolymers of uronic acids such as mannuronic acid, galatcuronic acid and guluronic acid, for example, alginic acid (a copolymer of beta-D-mannuronic acid and alpha-L-guluronic acid). Such charged polysaccharide species may be attached to a cell adhesion peptide, a protein, a protein fragment and/or a biocompatible polymer, as described in Ser. No. 10/781,932.

To the extent that the polymers of choice for use in the polymeric regions of the invention do not inherently have a charge, charged groups may nonetheless be introduced.

For example, as seen above in conjunction with sulfonated polymers, polymers including styrenic and other polymers may be chemically treated with various reagents, including reducing agents and oxidizing agents (e.g., sulfur trioxide for sulfonate formation), which modify their surfaces so as to provide them charged groups, such as such as amino, phosphate, sulfate, sulfonate, phosphonates and carboxylate groups.

As another example, charged groups may be introduced by covalently linking (grafting) charged species to the polymers. Examples of charged species include, for example, species containing the cationic and anionic groups discussed above. Covalent linkage may proceeds via a number of chemically reactive functional groups, including amino, hydroxyl, sulthydryl, carboxyl, and carbonyl groups, as well as carbohydrate groups, vicinal diols, thioethers, 2-aminoalcohols, 2-aminothiols, guanidinyl, imidazolyl and phenolic groups, among others.

Covalent coupling of charged species to polymers, each having reactive functional groups, may be carried out, for example, by direct reaction between such functional groups, or more typically by using linking agents that contain reactive moieties capable of reaction with such functional groups. Specific examples of commonly used linking agents include glutaraldehyde, diisocyanates, diiosothiocyanates, bis(hydroxysuccinimide)esters, maleimidehydroxysuccinimide esters, carbodiimides, N,N′-carbonyldiimidazole imidoesters, and difluorobenzene derivatives, among others. One ordinarily skilled in the art will recognize that any number of other coupling agents may be used depending on the functional groups present. In some embodiments, it is desirable for the polymer and the charged compound to have differing functional groups, so as to avoid self-coupling reactions. Functional groups present on the charged species and/or polymeric region may be converted, as desired, into other functional groups prior to reaction, e.g. to confer additional reactivity or selectivity. Further information on covalent coupling may be found, for example, in U.S. Pub. No. 2005/0002865, which is incorporated by reference.

As another example, charged groups may be introduced by non-covalently linking charged compounds to the polymers, for example, based on hydrogen bonding (e.g., multiple hydrogen bonds) between the polymers and with the charged compounds, based on the formation of complexes and/or coordinative bonds with charged species, or other strong non-covalent interaction.

As noted above, in certain embodiments, charge may be provided after the polymeric region is formed. For example, techniques such as those described above (e.g., chemical treatment, covalent or non-covalent coupling of charged species, etc.) may be performed on the polymeric region in order to provide charged groups.

Other techniques for providing surface charge include techniques whereby a polymeric region is treated with a reactive plasma. For example, gas discharge techniques have been used to functionalize polymer surfaces. Surface modification is obtained by exposing the surface to a partially ionized gas (i.e., to a plasma). Two types of processes are frequently described, depending on the operating pressure: corona discharge techniques (which are conducted at atmospheric pressure) and glow discharge techniques (which are conducted at reduced pressure). Because the plasma phase consists of a wide spectrum of reactive species (electrons, ions, etc.) these techniques have been used widely for functionalization of polymer surfaces.

Glow discharge techniques may be preferred over corona discharge techniques in certain embodiments, because the shape of the object to be treated is of minor importance during glow discharge processes. Moreover, glow discharge techniques are usually either operated in an etching or in a depositing mode, depending on the gas used, whereas corona discharge techniques are usually operated in an etching mode. A commonly employed glow discharge technique is radio-frequency glow discharge (RFGD).

Plasma treatment processes have been widely used to etch, crosslink and/or functionalize surfaces, with these processes occurring simultaneously at a polymer surface that is exposed to a discharge of a non-polymerizable gas. The gas that is used primarily determines which of these processes is dominant. When gases like carbon monoxide (CO), carbon dioxide (CO₂), or oxygen (O₂) are used, functionalization with —COOH groups (which donate protons to form anionic groups) is commonly observed. When gases like ammonia, a propyl amine, or N₂/H₂ are employed, —NH₂ groups (which accept protons to form cationic groups) are commonly formed.

Functional group containing surfaces may also be obtained using plasma polymerization processes in which “monomers” are employed that contain functional groups. Allylamine (which produces —NH₂ groups) and acrylic acid (which produces —COOH groups) have been used for this purpose. By using a second feed gas (generally a non-polymerizable gas) in combination with the unsaturated monomer, it is possible to incorporate this second species in the plasma deposited layer. Examples of gas pairs include allylamine/NH₃ (which leads to enhanced production of —NH₂ groups) and acrylic acid/CO₂ (which leads to enhanced production of —COOH groups).

The above and further information may be found, for example, in “Functionalization of Polymer Surfaces,” Europlasma Technical Paper, May 8, 2004 and in U.S. patent application Publication No. 2003/0236323.

In certain embodiments, charged polymeric regions in accordance with the invention are in the form of charged polymeric coatings. In these embodiments, the polymeric coatings may contain one or more polymer species that is charged (e.g., inherently or otherwise), or the polymeric coating may be processed to provide it with a surface charge (e.g., by chemical treatment, by covalent or non-covalent coupling of charged species, by plasma treatment), as detailed above.

Regardless of the method by which the polymeric region is created and provided with a charge, it is beneficial in certain embodiments to provide the polymeric region with a critical surface energy between 20 and 30 dynes/cm. Surfaces having a critical surface energy between 20-30 dynes/cm have been shown in work by Dr. Robert Baier and others to provide enhanced biocompatibility, including enhanced thromboresistance. See, e.g., Baier R E, Meenaghan M A, Hartman L C, Wirth J E, Flynn H E, Meyer A E, Natiella J R, Carter J M, “Implant Surface Characteristics and Tissue Interaction”, J Oral Implantol, 1988, 13(4), 594-606; Robert Baier, Joseph Natiella, Anne Meyer, John Carter, “Importance of Implant Surface Preparation for Biomaterials with Different Intrinsic Properties in Tissue Integration in Oral and Maxillofacial Reconstruction”; Current Clinical Practice Series #29, 1986; Robert Baier, Joseph Natiella, Anne Meyer, John Carter, Formalik, M. S., Tumbull, T., “Surface Phenomena in In Vivo Environments. Applications of Materials Sciences to the Practice of Implant Orthopedic Surgery”, NATO Advanced Study Institute, Costa Del Sol, Spain, 1984; Baier R E, Meyer A E, Natiella J R, Natiella R R, Carter J M, “Surface properties determine bioadhesive outcomes: methods and results”, J Biomed Mater Res, 1984, 18(4), 327-355; Joseph Natiella, Robert Baier, John Carter, Anne Meyer, Meenaghan, M. A., Flynn, H. E., “Differences in Host Tissue Reactions to Surface-Modified Dental Implants”, 185th ACS National Meeting, American Chemical Society, 1983.

In this regard, methods are known for measuring critical surface energy. For example, contact angle methods can be used to produce Zisman plots for calculating critical surface tensions. For further information on measuring critical surface energy, see, e.g., Zisman, W. A., “Relation of the equilibrium contact angle to liquid and solid constitution,” Adv. Chem. Ser. 43, 1964, pp. 1-51; Baier R. E., Shiafrin E. G., Zisman, W. A., “Adhesion: Mechanisms that assist or impede it,” Science, 162: 1360-1368, 1968; Fowkes, F. M., “Contact angle, wettability and adhesion,” Washington D.C., Advances in Chemistry, vol. 43, 1964, p. 1, Souheng Wu, Polymer Interface and Adhesion, Marcel Dekker, 1982, Chapter 5, pp. 169-212. Hence, various polymer surfaces may be tested, if desired, to determine whether or not those surfaces have a critical surface energy within the above criteria.

Numerous techniques are available for forming polymeric regions in accordance with the present invention. For example, wherein one or more polymers within the polymeric regions have thermoplastic characteristics, and so long as the polymers and any other optional supplemental materials are sufficiently stable under processing conditions, a variety of standard thermoplastic processing techniques may be used to form the polymeric regions, including compression molding, injection molding, blow molding, spinning, vacuum forming, calendaring, extrusion into sheets, fibers, rods, tubes and other cross-sectional profiles of various lengths, and coextrusion into multilayered structures. Using these and other thermoplastic processing techniques, entire medical articles or portions thereof can be made.

In other embodiments, solvent-based techniques may be used to form polymeric regions in accordance with the present invention. Using these techniques, polymeric regions may be formed by first providing solutions that contain the one or more polymers of interest (and any other optional supplemental materials to be processed), and subsequently removing the solvents to form the polymeric regions. The solvents that are ultimately selected will contain one or more solvent species, which are generally selected based on their ability to dissolve the materials that form the polymeric region, as well as other factors, including drying rate, surface tension, etc. Solvent-based techniques include, but are not limited to, solvent casting techniques, spin coating techniques, web coating techniques, fiber spinning techniques, solvent spraying techniques, dipping techniques, techniques involving coating via mechanical suspension including air suspension, ink jet techniques, electrostatic techniques, and combinations of these processes.

In some embodiments of the invention, a solution (where solvent-based processing is employed) or a melt (where thermoplastic processing is employed) is applied to a substrate to form a polymeric region. For example, the substrate can correspond to all or a portion of an implantable or insertable medical device to which a polymeric region is applied. The substrate can also be, for example, a template, such as a mold, from which the polymeric region is removed after solidification. In other embodiments, for example, extrusion and co-extrusion techniques, one or more polymeric regions are formed without the aid of a substrate.

Taking fibers as a specific example, where employed, they may be made by any suitable fiber forming technique, including melt spinning, dry spinning and wet spinning. These processes typically employ extrusion nozzles having one or more orifices, called jets or spinnerets. Fibers having a variety of cross-sectional shapes can be formed, depending upon the shape of the orifice(s) in the spinning die. Some examples of fiber cross-sections include circular, hexagonal, rectangular, triangular, oval, multi-lobed, and annular (hollow) cross-sections. In melt spinning, the polymer compound is heated to melt temperature. In wet and dry spinning the polymer is dissolved in a solvent prior to extrusion, and the extrudate is subjected to conditions whereby the solvent is evaporated, for example, by exposure to a vacuum or heated atmosphere (e.g., air) which removes the solvent by evaporation. In wet spinning the jet or spinneret is immersed in a liquid, and as the extrudate emerges, it precipitates from solution and solidifies. Regardless of the technique, the resulting fiber is typically taken up on a rotating mandrel or another take-up device. During take up, the fiber may be stretched to orient the polymer molecules.

In accordance with certain embodiments of the present invention, a dry spinning technique is employed in which a styrene-isobutylene copolymer containing solution is fed (e.g., using a metering pump such as a syringe pump) through one or more fine orifices (e.g., those found in a dry spinning die, or spinneret). Further details regarding dry spinning of styrene-isobutylene copolymers, may be found in copending, commonly assigned U.S. Ser. No. 10/801,228.

As indicated above, fibers may be formed into two- and three-dimensional medical articles. One particularly beneficial method for forming porous tubular three-dimensional structures from fibers is described in U.S. Pat. No. 4,475,972 to Wong, the disclosure of which is hereby incorporated by reference, in which these articles are made by a procedure in which fibers are wound on a mandrel and overlying fiber portions are simultaneously bonded with underlying fiber portions. For instance, a polymer solution may be extruded from a spinneret, thereby forming a plurality of filaments which are wound onto a rotating mandrel, as the spinneret reciprocates relative to the mandrel. The drying parameters (e.g., drying environment, solution temperature and concentration, spinneret-to-mandrel distance, etc.) are controlled such that some residual solvent remains in the filaments as they are wrapped upon the mandrel. Upon further evaporation of the solvent, the overlapping fibers on the mandrel become bonded to each other.

As discussed in detail above, fibers may be provided with charges via a variety of techniques, including forming the fibers using charged polymers (e.g., those that are inherently charged or are provided with a charge before fiber formation) or by treating the fiber subsequent to its formation (e.g., by chemical treatment with a species that produces charged groups such as oxidizing and reducing agents, by covalent or non-covalent coupling of charged species, by plasma treatment, etc.), for example, either before or after being formed into a two- or three-dimensional article. For instance, the styrene-isobutylene copolymer described in commonly assigned U.S. Ser. No. 10/801,228 may be provided with a charge before fiber spinning (e.g., by sulfonation, etc.) or after fiber spinning (e.g., by plasma treatment, sulfonation, etc.).

An advantage to providing medical articles with charged polymeric regions is that, by selecting a charged therapeutic agent of opposite charge, binding between the polymeric region and the therapeutic agent may be enhanced. “Therapeutic agents,” drugs,” “bioactive agents” “pharmaceuticals,” “pharmaceutically active agents”, and other related terms may be used interchangeably herein and include genetic and non-genetic therapeutic agents as well as cells. Therapeutic agents may be used singly or in combination.

A wide range of therapeutic agent loadings can be used in conjunction with the devices of the present invention, with the pharmaceutically effective amount being readily determined by those of ordinary skill in the art and ultimately depending, for example, upon the condition to be treated, the nature of the therapeutic agent itself, the tissue into which the dosage form is introduced, and so forth.

Exemplary non-genetic biologically active agents for use in connection with the present invention include: (a) anti-thrombotic agents such as heparin, heparin derivatives, urokinase, and PPack (dextrophenylalanine proline arginine chloromethylketone); (b) anti-inflammatory agents such as dexamethasone, prednisolone, corticosterone, budesonide, estrogen, sulfasalazine and mesalamine; (c) antineoplastic/antiproliferative/anti-miotic agents such as paclitaxel, 5-fluorouracil, cisplatin, vinblastine, vincristine, epothilones, endostatin, angiostatin, angiopeptin, monoclonal antibodies capable of blocking smooth muscle cell proliferation, and thymidine kinase inhibitors; (d) anesthetic agents such as lidocaine, bupivacaine and ropivacaine; (e) anti-coagulants such as D-Phe-Pro-Arg chloromethyl ketone, an RGD peptide-containing compound, heparin, hirudin, antithrombin compounds, platelet receptor antagonists, anti-thrombin antibodies, anti-platelet receptor antibodies, aspirin, prostaglandin inhibitors, platelet inhibitors and tick antiplatelet peptides; (f) vascular cell growth promoters such as growth factors, transcriptional activators, and translational promotors; (g) vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; (h) protein kinase and tyrosine kinase inhibitors (e.g., tyrphostins, genistein, quinoxalines); (i) prostacyclin analogs; (j) cholesterol-lowering agents; (k) angiopoietins; (l) antimicrobial agents such as triclosan, cephalosporins, antimicrobial peptides such as magainins, aminoglycosides and nitrofurantoin; (m) cytotoxic agents, cytostatic agents and cell proliferation affectors; (n) vasodilating agents; (o) agents that interfere with endogenous vasoactive mechanisms, (p) inhibitors of leukocyte recruitment, such as monoclonal antibodies; (q) cytokines; (r) hormones; (s) inhibitors of HSP 90 protein (i.e., Heat Shock Protein, which is a molecular chaperone or housekeeping protein and is needed for the stability and function of other client proteins/signal transduction proteins responsible for growth and survival of cells) including geldanamycin, (t) beta-blockers, (u) bARKct inhibitors, (v) phospholamban inhibitors, (w) Serca 2 gene/protein, (x) immune response modifiers including aminoquizolines, for instance, imidazoquinolines such as resiquimod and imiquimod, (y) human apolioproteins (e.g., AI, AII, AIII, AIV, AV, etc.).

Some preferred non-genetic therapeutic agents include paclitaxel (including particulate forms thereof, for instance, protein-bound paclitaxel particles such as albumin-bound paclitaxel nanoparticles, e.g., ABRAXANE), sirolimus, everolimus, tacrolimus, Epo D, dexamethasone, estradiol, halofuginone, cilostazole, geldanamycin, ABT-578 (Abbott Laboratories), trapidil, liprostin, Actinomcin D, Resten-NG, Ap-17, abciximab, clopidogrel, and Ridogrel, beta-blockers, bARKct inhibitors, phospholamban inhibitors, Serca 2 gene/protein, imiquimod, human apolioproteins (e.g., AI-AV), growth factors (e.g., VEGF-2), as well a derivatives of the forgoing, among others.

Exemplary genetic biologically active agents for use in connection with the present invention include anti-sense DNA and RNA as well as DNA coding for: (a) anti-sense RNA, (b) tRNA or rRNA to replace defective or deficient endogenous molecules, (c) angiogenic factors including growth factors such as acidic and basic fibroblast growth factors, vascular endothelial growth factor, epidermal growth factor, transforming growth factor α and β, platelet-derived endothelial growth factor, platelet-derived growth factor, tumor necrosis factor α, hepatocyte growth factor and insulin-like growth factor, (d) cell cycle inhibitors including CD inhibitors, and (e) thymidine kinase (“TK”) and other agents useful for interfering with cell proliferation. Also of interest is DNA encoding for the family of bone morphogenic proteins (“BMP's”), including BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15, and BMP-16. Currently preferred BMP's are any of BMP-2, BMP-3, BMP-4, BMP-5, BMP-6 and BMP-7. These dimeric proteins can be provided as homodimers, heterodimers, or combinations thereof, alone or together with other molecules. Alternatively, or in addition, molecules capable of inducing an upstream or downstream effect of a BMP can be provided. Such molecules include any of the “hedgehog” proteins, or the DNA's encoding them.

Vectors for delivery of genetic therapeutic agents include viral vectors such as adenoviruses, gutted adenoviruses, adeno-associated virus, retroviruses, alpha virus (Semliki Forest, Sindbis, etc.), lentiviruses, herpes simplex virus, replication competent viruses (e.g., ONYX-015) and hybrid vectors; and non-viral vectors such as artificial chromosomes and mini-chromosomes, plasmid DNA vectors (e.g., pCOR), cationic polymers (e.g., polyethyleneimine, polyethyleneimine (PEI)), graft copolymers (e.g., polyether-PEI and polyethylene oxide-PEI), neutral polymers PVP, SP1017 (SUPRATEK), lipids such as cationic lipids, liposomes, lipoplexes, nanoparticles, or microparticles, with and without targeting sequences such as the protein transduction domain (PTD).

Cells for use in connection with the present invention include cells of human origin (autologous or allogeneic), including whole bone marrow, bone marrow derived mono-nuclear cells, progenitor cells (e.g., endothelial progenitor cells), stem cells (e.g., mesenchymal, hematopoietic, neuronal), pluripotent stem cells, fibroblasts, myoblasts, satellite cells, pericytes, cardiomyocytes, skeletal myocytes or macrophage, or from an animal, bacterial or fungal source (xenogeneic), which can be genetically engineered, if desired, to deliver proteins of interest.

Numerous biologically active agents, not necessarily exclusive of those listed above, have been identified as candidates for vascular treatment regimens, for example, as agents targeting restenosis. Such agents are useful for the practice of the present invention and include one or more of the following: (a) Ca-channel blockers including benzothiazapines such as diltiazem and clentiazem, dihydropyridines such as nifedipine, amlodipine and nicardapine, and phenylalkylamines such as verapamil, (b) serotonin pathway modulators including: 5-HT antagonists such as ketanserin and naftidrofuryl, as well as 5-HT uptake inhibitors such as fluoxetine, (c) cyclic nucleotide pathway agents including phosphodiesterase inhibitors such as cilostazole and dipyridamole, adenylate/Guanylate cyclase stimulants such as forskolin, as well as adenosine analogs, (d) catecholamine modulators including α-antagonists such as prazosin and bunazosine, β-antagonists such as propranolol and α/β-antagonists such as labetalol and carvedilol, (e) endothelin receptor antagonists, (f) nitric oxide donors/releasing molecules including organic nitrates/nitrites such as nitroglycerin, isosorbide dinitrate and amyl nitrite, inorganic nitroso compounds such as sodium nitroprusside, sydnonimines such as molsidomine and linsidomine, nonoates such as diazenium diolates and NO adducts of alkanediamines, S-nitroso compounds including low molecular weight compounds (e.g., S-nitroso derivatives of captopril, glutathione and N-acetyl penicillamine) and high molecular weight compounds (e.g., S-nitroso derivatives of proteins, peptides, oligosaccharides, polysaccharides, synthetic polymers/oligomers and natural polymers/oligomers), as well as C-nitroso-compounds, O-nitroso-compounds, N-nitroso-compounds and L-arginine, (g) Angiotensin Converting Enzyme (ACE) inhibitors such as cilazapril, fosinopril and enalapril, (h) ATII-receptor antagonists such as saralasin and losartin, (i) platelet adhesion inhibitors such as albumin and polyethylene oxide, (j) platelet aggregation inhibitors including cilostazole, aspirin and thienopyridine (ticlopidine, clopidogrel) and GP IIb/IIIa inhibitors such as abciximab, epitifibatide and tirofiban, (k) coagulation pathway modulators including heparinoids such as heparin, low molecular weight heparin, dextran sulfate and β-cyclodextrin tetradecasulfate, thrombin inhibitors such as hirudin, hirulog, PPACK(D-phe-L-propyl-L-arg-chloromethylketone) and argatroban, FXa inhibitors such as antistatin and TAP (tick anticoagulant peptide), Vitamin K inhibitors such as warfarin, as well as activated protein C, (l) cyclooxygenase pathway inhibitors such as aspirin, ibuprofen, flurbiprofen, indomethacin and sulfinpyrazone, (m) natural and synthetic corticosteroids such as dexamethasone, prednisolone, methprednisolone and hydrocortisone, (n) lipoxygenase pathway inhibitors such as nordihydroguairetic acid and caffeic acid, (o) leukotriene receptor antagonists, (p) antagonists of E- and P-selectins, (q) inhibitors of VCAM-1 and ICAM-1 interactions, (r) prostaglandins and analogs thereof including prostaglandins such as PGE1 and PGI2 and prostacyclin analogs such as ciprostene, epoprostenol, carbacyclin, iloprost and beraprost, (s) macrophage activation preventers including bisphosphonates, (t) HMG-CoA reductase inhibitors such as lovastatin, pravastatin, fluvastatin, simvastatin and cerivastatin, (u) fish oils and omega-3-fatty acids, (v) free-radical scavengers/antioxidants such as probucol, vitamins C and E, ebselen, trans-retinoic acid and SOD mimics, (w) agents affecting various growth factors including FGF pathway agents such as bFGF antibodies and chimeric fusion proteins, PDGF receptor antagonists such as trapidil, IGF pathway agents including somatostatin analogs such as angiopeptin and ocreotide, TGF-β pathway agents such as polyanionic agents (heparin, fucoidin), decorin, and TGF-β antibodies, EGF pathway agents such as EGF antibodies, receptor antagonists and chimeric fusion proteins, TNF-α pathway agents such as thalidomide and analogs thereof, Thromboxane A2 (TXA2) pathway modulators such as sulotroban, vapiprost, dazoxiben and ridogrel, as well as protein tyrosine kinase inhibitors such as tyrphostin, genistein and quinoxaline derivatives, (x) MMP pathway inhibitors such as marimastat, ilomastat and metastat, (y) cell motility inhibitors such as cytochalasin B, (z) antiproliferative/antineoplastic agents including antimetabolites such as purine analogs (e.g., 6-mercaptopurine or cladribine, which is a chlorinated purine nucleoside analog), pyrimidine analogs (e.g., cytarabine and 5-fluorouracil) and methotrexate, nitrogen mustards, alkyl sulfonates, ethylenimines, antibiotics (e.g., daunorubicin, doxorubicin), nitrosoureas, cisplatin, agents affecting microtubule dynamics (e.g., vinblastine, vincristine, colchicine, Epo D, paclitaxel and epothilone), caspase activators, proteasome inhibitors, angiogenesis inhibitors (e.g., endostatin, angiostatin and squalamine), rapamycin, cerivastatin, flavopiridol and suramin, (aa) matrix deposition/organization pathway inhibitors such as halofuginone or other quinazolinone derivatives and tranilast, (bb) endothelialization facilitators such as VEGF and RGD peptide, and (cc) blood rheology modulators such as pentoxifylline.

Numerous additional biologically active agents useful for the practice of the present invention are also disclosed in U.S. Pat. No. 5,733,925 assigned to NeoRx Corporation, the entire disclosure of which is incorporated by reference.

Therapeutic agents for use in the present invention have an associated charge. For example, a therapeutic agent may have an associated charge because it is inherently charged (e.g., because it has acidic and/or or basic groups, which may be in salt form). A few examples of inherently charged cationic therapeutic agents include amiloride, digoxin, morphine, procainamide, and quinine, among many others. Examples of anionic therapeutic agents include DNA, among many others.

As another example, a therapeutic agent may have an associated charge because it has been modified to carry a charge, for example, by covalently or non-covalently coupling a charged species to the therapeutic agent.

For instance, various cationic forms of paclitaxel are known, including paclitaxel methylpyridinium mesylate and paclitaxel conjugated with N-2-hydroxypropyl methyl amide, as are various anionic forms of paclitaxel, including paclitaxel-poly(1-glutamic acid), paclitaxel-poly(1-glutamic acid)-PEO. In addition to these, U.S. Pat. No. 6,730,699, which is incorporated by reference in its entirety, also describes paclitaxel conjugated to various other charged polymers including poly(d-glutamic acid), poly(dl-glutamic acid), poly(1-aspartic acid), poly(d-aspartic acid), poly(dl-aspartic acid), poly(1-lysine), poly(d-lysine), poly(dl-lysine), copolymers of the above listed polyamino acids with polyethylene glycol, polycaprolactone, polyglycolic acid and polylactic acid, as well as poly(2-hydroxyethyl 1-glutamine), chitosan, carboxymethyl dextran, hyaluronic acid, human serum albumin and alginic acid. Still other anionic forms of paclitaxel include carboxylated forms such as 1′-malyl paclitaxel sodium salt (see, e.g. E. W. DAmen et al., “Paclitaxel esters of malic acid as prodrugs with improved water solubility,” Bioorg Med. Chem., 2000 Feb., 8(2), pp. 427-32), which is incorporated by reference in its entirety.

As yet another example, a therapeutic agent may have an associated charge because it is attached to or encapsulated within a charged particle, for example, a charged nanoparticle (i.e., a charged particle having a cross-sectional dimension of 100 nm or less, for example, a spherical particle or a rod-shaped particle having a diameter of 100 nm or less) such as a nanocapsule or a charged micelle, among others.

Charged nanoparticles may be formed, for example, from polycationic polymers and polyanionic polymers such as those described above. Specific examples of charged nanoparticles include nanocapsules that contain alternating layers of (a) a polyanion, for example, poly(L-glutamic acid) and (b) a polycation, for example, poly(L-lysine). If desired a charged or uncharged therapeutic agent may be provided within the core of the nanocapsule. For example, see I. L. Radtchenko et al., “A novel method for encapsulation of poorly water-soluble drugs: precipitation in polyelectrolyte multilayer shells,” International Journal of Pharmaceutics, 242 (2002) 219-223 which is incorporated by reference in its entirety. Charged therapeutic agent (e.g., paclitaxel conjugated to a polycation such as poly-L-lysine or a polyanion such as poly-L-glutamic acid, among many others) may also be provided within one or more of the layers of the nanocapsule.

Further specific examples of charged particles include charged micelles. For example, poly(ethylene oxide)-block-poly(amino acids) may form charged micelles for drug delivery.

Polymeric regions in accordance with the present invention may be loaded with therapeutic agents, for example, at various points after their formation. For example, taking fibrous articles as an example, therapeutic agent loading may occur after fiber formation or after the fiber is shaped into a medical article (e.g., by a woven process, a non-woven process, etc.). Loading may be conducted, for example, by exposing polymeric regions and therapeutic agents of opposite charge to one another. For example, the charged polymeric regions and therapeutic agents may be exposed to one another in an aqueous solution at a pH where the therapeutic agent and the polymeric region and have opposite charges.

Although various embodiments are specifically illustrated and described herein, it will be appreciated that modifications and variations of the present invention are covered by the above teachings and are within the purview of the appended claims without departing from the spirit and intended scope of the invention.

What is claimed is:

1. A medical article comprising (a) a fiber having a diameter of 10 microns or less, said fiber comprising a polymeric region having a first charge, and (b) a charged therapeutic agent having a second charge that is opposite in sign to that of said first charge that is bound to said fiber.

2. The medical article of claim 1, wherein said medical article comprises a plurality of fibers.

3. The medical article of claim 1, wherein said fiber comprises a plurality of polymeric regions.

4. The medical article of claim 1, wherein a plurality of different charged therapeutic agents are bound to said fiber.

5. The medical article of claim 1, wherein said polymeric region extends throughout the diameter of said fiber.

6. The medical article of claim 1, wherein said polymeric region is a coating layer.

7. The medical article of claim 1, wherein said polymeric region comprises a nanotextured surface.

8. The medical article of claim 1, wherein said polymeric region has a critical surface energy between 20 and 30 dynes/cm.

9. The medical article of claim 1, wherein polymers make up 75% or more of said polymeric region.

10. The medical article of claim 1, wherein said fiber has a diameter between 10 nm and 10 microns.

11. The medical article of claim 1, wherein said first charge is a positive charge and said second charge is a negative charge.

12. The medical article of claim 11, wherein said charged therapeutic agent is DNA.

13. The medical article of claim 1, wherein said first charge is a negative charge and said second charge is a positive charge.

14. The medical article of claim 13, wherein said therapeutic agent is selected from amiloride, digoxin, morphine, procainamide, and quinine.

15. The medical article of claim 1, wherein said polymeric region comprises a polyionic polymer.

16. The medical article of claim 15, wherein said polyionic polymer is a polycationic polymer.

17. The medical article of claim 15, wherein said polyionic polymer is a polyanionic polymer.

18. The medical article of claim 1, wherein a charged species having said first charge is covalently attached to a surface of said polymeric region.

19. The medical article of claim 18, wherein said charged species is linked to said polymeric region using a linking agent.

20. The medical article of claim 18, wherein said charged species is provided by chemical treatment.

21. The medical article of claim 20, wherein said polymeric region is treated with an oxidizing or reducing agent.

22. The medical article of claim 20, wherein said surface comprises sulfonate groups.

23. The medical article of claim 20, wherein said chemical treatment comprises plasma treatment in the presence of a reactive gas.

24. The medical article of claim 23, wherein said gas comprises oxygen, carbon monoxide, carbon dioxide, ammonia, a mixture of molecular hydrogen and molecular nitrogen, or an amine.

25. The medical article of claim 23, wherein said gas comprises an unsaturated species that further comprises an acidic or basic functional group.

26. The medical article of claim 1, wherein a charged species having said first charge is non-covalently linked to a surface of said polymeric region.

27. The medical article of claim 1, wherein said charged therapeutic agent is selected from a therapeutic agent that is inherently charged, a therapeutic agent that is covalently attached to a charged molecule, a therapeutic agent that is non-covalently attached to a charged molecule, a therapeutic agent that is attached to or encapsulated within a charged particle, and a combination thereof.

28. The medical article of claim 1, wherein the polymeric region comprises a copolymer that comprises styrene and isobutylene.

29. The medical article of claim 28, wherein the copolymer comprises a polyisobutylene block and a polystyrene block.

30. The medical article of claim 28, wherein the copolymer is a polystyrene-polyisobutylene-polystyrene triblock copolymer.

31. The medical article of claim 28, wherein the copolymer is sulfonated.

32. The medical article of claim 28, wherein said medical article comprises a woven region comprising said fiber.

33. The medical article of claim 1, wherein said medical article comprises a non-woven region comprising said fiber.

34. The medical article of claim 1, wherein said medical article is a porous, tubular medical article.

35. The medical article of claim 1, wherein said medical article is selected from hollow fibers for oxygenators, hernia repair patches, gastrointestinal tract patches, uro-gynecological tract patches, vascular access ports, fabric to join devices to human arteries, wound dressings, membranes, anterior cruciate ligaments, neurovascular aneurysm treatment articles, valve leaflets for heart valves, valve leaflets for venous valves, stents, stent grafts, gastrointestinal tract grafts, uro-gynecological tract grafts, coronary vascular grafts, peripheral vascular grafts, arterio-venous access grafts, embolic filters, and scaffolds for tissue engineering.

36. The medical article of claim 1, wherein said medical article comprises a region comprising said fiber, disposed over an underlying medical device substrate.

37. The medical article of claim 36, wherein said medical device substrate is a metallic stent.
